(19) United States
(12) Patent Application Publication
(10) Pub. No.: US 2022/0112460 A1
(43) Pub. Date: Apr. 14, 2022

(54) NANOCOIL-SUBSTRATE COMPLEX FOR CONTROLLING STEM CELL BEHAVIOR, PREPARATION METHOD THEREOF, AND METHOD OF CONTROLLING ADHESION AND DIFFERENTIATION OF STEM CELL BY USING THE SAME

(71) Applicant: Korea University Research and Business Foundation, Seoul (KR)

(72) Inventors: Young-Keun KIM, Seoul (KR); Heemin KANG, Seoul (KR); Min-Jun KO, Seoul (KR); Sun-Hong MIN, Suwon-si (KR)

(73) Assignee: Korea University Research and Business Foundation, Seoul (KR)

(21) Appl. No.: 17/443,501

(22) Filed: Jul. 27, 2021

(30) Foreign Application Priority Data
Oct. 13, 2020 (KR) 10-2020-0131889

(51) Int. Cl. C12N 5/074; C07K 14/705; B82Y 30/00; B82Y 5/00; B82Y 40/00; C25D 1/00; C25D 1/04

(52) U.S. Cl. CPC C12N 5/0607; C07K 14/70546; B82Y 30/00; B82Y 5/00; C25D 3/12; C25D 1/006; C25D 1/04; C12N 2529/00; B82Y 40/00

(57) ABSTRACT

The present invention relates to a nanocoil-substrate complex for controlling adhesion and differentiation of stem cells, a manufacturing method thereof, and a method of controlling adhesion and differentiation of stem cells by using the nanocoil-substrate complex, and the method of controlling adhesion and differentiation of stem cells may temporally and reversibly control adhesion and phenotypic differentiation of stem cells in vivo and ex vivo by controlling application/non-application of a magnetic field to the nanocoil-substrate complex.

### CROSS-REFERENCE TO RELATED APPLICATION

This application claims a benefit under 35 U.S.C. § 119(a) of Korean Patent Application No. 10-2020-0131889 filed on Oct. 13, 2020, on the Korean Intellectual Property Office, the entire disclosure of which is incorporated herein by reference for all purposes.

### TECHNICAL FIELD

The present invention relates to a nanocoil-substrate complex for controlling adhesion and differentiation of stem cells, a preparation method thereof, and a method of controlling adhesion and differentiation of stem cells by using the nanocoil-substrate complex, and particularly, to a method of controlling cell adhesion and differentiation of stem cells depending on application/non-application of a magnetic field to the nanocoil-substrate complex.

### BACKGROUND ART

Stem cells can proliferate through self-renewal, and have the potential to differentiate into various cells, such as bone, fat, muscle, myocardium, blood vessels, and cartilage. Recently, in order to regenerate damaged tissues and organs by using these characteristics, many studies have been conducted on transplantation of stem cells or cells differentiated from stem cells. In addition, biomaterials that can help stem cells to differentiate into specific cells are also being actively studied.

As a method of efficiently controlling the regenerative effect of stem cells, a technology through the presentation of ligand in vivo is used. However, there is a problem in that the existing micro-scale integrin ligand peptide (RGD) uncaging controls the adhesion of host stem cells, but does not control the differentiation of stem cells.

In this respect, the present applicant developed the technology of controlling adhesion and differentiation of stem cells by controlling periodicity and sequences of nanobarcode ligands and filed the technology for a patent application.

In addition, the applicant of the present application intends to propose a technology that is capable of providing a more improved and bio-friendly technology compared to the previously filed stem cell adhesion and differentiation control technology below, and particularly, intends to propose a technology that is capable of changing a characteristic of cells in real time by using external stimuli after injection, rather than a method of designing and inserting ligands in advance.

### PRIOR ART LITERATURE

(Patent Document) Korean Patent No. 10-1916588

### SUMMARY OF THE INVENTION

The present invention is conceived to solve the foregoing problems, and is to provide a substrate including ligand-coated nanocoils, and a method of controlling adhesion and differentiation of stem cells by controlling an application of a magnetic field to the ligand-coated nanocoils.

The present invention provides a nanocoil-substrate complex for controlling adhesion and differentiation of stem cells, the nanocoil-substrate complex including: a substrate; one or more nanocoils chemically coupled to the substrate; and one or more integrin ligand peptides chemically coupled to the nanocoil, in which the nanocoil is formed of a spiral nanowire and includes one or more metal elements, the nanocoil has a length of 100 nm to 20 μm, and the nanocoil has a length reversibly changed depending on application/non-application of a magnetic field within a range of Equation 1 below.

|L₁−L₀|>10 nm [Equation 1]

In Equation 1, L₁ is a length of the nanocoil when the magnetic field is applied, and L₀ is a length of the nanocoil when the magnetic field is not applied.

Further, the present invention provides a method of preparing a nanocoil-substrate complex for controlling adhesion and differentiation of stem cells, the method including: preparing a nanocoil by electrodepositing a solution including one or more metal elements; coupling a carboxylate substituent to the nanocoil by mixing the nanocoil and a first suspension; manufacturing a substrate coupled with the nanocoil by soaking a substrate, of which a surface is activated, in a solution containing the nanocoil to which the carboxylate is coupled; coupling a linker to a distal end of the nanocoil by soaking the substrate coupled with the nanocoil in a solution containing a polyethylene glycol linker; and coupling an integrin ligand peptide (RGD) to the nanocoil by mixing a second suspension containing the integrin ligand peptide and the activated substrate coupled with the nanocoil.

Furthermore, the present invention provides a method of controlling adhesion and differentiation of stem cells, the method including: controlling cell adhesion and differentiation of stem cells by treating the nanocoil-substrate complex for controlling cell adhesion and differentiation of the stem cells with a culture medium and then applying a magnetic field in a range from 20 mT to 7 T, in which the nanocoil has a length reversibly changed within Equation 1 below depending on application/non-application of the magnetic field.

|L₁−L₀|>10 nm [Equation 1]

In Equation 1, L₁ is a length of the nanocoil when the magnetic field is applied, and L₀ is a length of the nanocoil when the magnetic field is not applied.

The nanocoil-substrate complex for controlling adhesion and differentiation of stem cells according to the present invention may reversibly control adhesion and differentiation by controlling the application/non-application of a magnetic field to the nanocoil coated with the integrin ligand, and efficiently adjust adhesion and phenotypic differentiation of stem cells in vivo and ex vivo.

### BRIEF DESCRIPTION OF THE DRAWINGS

FIG. 1 is a schematic diagram illustrating a nanocoil-substrate complex for controlling cell adhesion and differentiation of stem cells and a method of controlling adhesion and differentiation of stem cells by using the same according to an exemplary embodiment of the present invention.

FIG. 2 is a scanning electron microscope image of a nanocoil according to the present invention.

FIG. 3 is a High-Angle Annular Dark Field Scanning Transmission Electron Microscope (HAADF-STEM) image, a Scanning Electron Microscope (SEM) image, an Energy Dispersive Spectroscopy (EDS) mapping image, and a High-Resolution Scanning Transmission Electron Microscopy (HR-STEM) image of the nanocoil according to the present invention, a scale bar of the HAADF-STEM represent 250 nm, a scale bar of the SEM represents 1 μm, and a scale bar of the HR-STEM represents 4 Å.

FIG. 4 is a graph illustrating an EDS analysis result, and a graph and a mapping image illustrating an EELS analysis result of the nanocoil according to the present invention, and a scale bar represents 200 nm.

FIG. 5 is a High-Resolution Transmission Electron Microscopy (HRTEM) image of the nanocoil according to the present invention, the left scale bar represents 300 nm, and the right scale bar represents 2 nm.

FIG. 6 is an X-ray diffraction analysis graph of the nanocoil according to the present invention,

FIG. 7 is a graph illustrating a vibrating-sample magnetometry measurement result of the nanocoil according to the present invention.

FIG. 8 is an image schematically illustrating an operation of preparing a nanocoil-substrate complex according to the present invention.

FIG. 9 is a diagram illustrating a result of a Fourier Transform Infrared Spectroscopy (FT-IR) analysis of the nanocoil-substrate complex according to the present invention.

FIGS. 10 and 11 are an Atomic Force Microscope (AFM) images and a graph representing the length of the nanocoil according to the present invention and a scale bar represents 500 nm.

FIG. 12 is a confocal immunofluorescent image of F-actin, nuclei, and vinculin in stem cells cultured (after 48 hours) by using the nanocoil-substrate complex according to the present invention, and a graph illustrating an adherent cell density, a cell area, focal adherence number, and an aspect ratio (a ratio of major axis/minor axis) calculated based on the result of the confocal immunofluorescent experiment, and a scale bar represents 50 μm.

FIG. 13 is a confocal immunofluorescent image of F-actin, nuclei, and vinculin in stem cells cultured (after 54 hours) by changing an application of a magnetic field every 18 hours by using the nanocoil-substrate complex according to the present invention, and a scale bar represents 50 μm.

FIG. 14 is a graph illustrating an adherent cell density, a cell area, focal adherence number, and an aspect ratio (a ratio of major axis/minor axis) of the stem cells cultured by changing the application of a magnetic field at an interval of 18 hours by using the nanocoil-substrate complex according to the present invention calculated based on the result of the confocal immunofluorescent experiment.

FIG. 15 is a confocal immunofluorescent image of live cells and dead cells in stem cells cultured (after 48 hours) by using the nanocoil-substrate complex according to the present invention, and a graph illustrating cell viability calculated based on the result of the confocal immunofluorescent experiment, and a scale bar represents 50 μm.

FIG. 16 is a diagram illustrating a result of an experiment for adhesion of stem cells for bimodal switching in a substrate having no nanocoil or a nanocoil-substrate complex to which the integrin ligand (RGD) is not coupled according to a comparative example of the present invention.

FIG. 17 is a confocal immunofluorescent image of F-actin, nuclei, and vinculin in stem cells cultured for 36 hours by adjusting an application of a magnetic field at an interval of 18 hours by using the nanocoil-substrate complex according to the present invention, and a graph illustrating nuclear/cytoplasmic YAP ratio calculated based on the result of the confocal immunofluorescent experiment, and a scale bar represents 50 μm.

FIG. 18 is a result of the confocal immunofluorescent analysis for F-actin, nuclei, and TAZ in stem cells cultured for 36 hours by adjusting an application of a magnetic field at an interval of 18 hours by using the nanocoil-substrate complex according to the present invention.

FIG. 19 is a result of the confocal immunofluorescent analysis for osteocalcin, F-actin, nuclei in stem cells cultured 5 days by using the nanocoil-substrate complex according to the present invention, in which an application of a magnetic field is adjusted at the second day.

FIG. 20 is a graph illustrating a quantitative analysis of the nuclear/cytoplasmic RUNX2 and ALP gene expression profile in stem cells cultured for 3 days by using the nanocoil-substrate complex according to the present invention, in which an application of a magnetic field is adjusted after one day.

FIG. 21 is a result of the confocal immunofluorescent analysis for ALP genes, RUNX2, F-actin, and nuclei in stem cells cultured 5 days by using the nanocoil-substrate complex according to the present invention, in which an application of a magnetic field is adjusted at the second day.

FIG. 22 is a result of the confocal immunofluorescent analysis for YAP, F-actin, and nuclei in stem cells cultured for 48 hours in a medium without an inhibitor and a medium with ROCK inhibitor (Y27632) and myosin II inhibitor (blebbistatin) by using the nanocoil-substrate complex according to the present invention.

FIG. 23 is a result of the confocal immunofluorescent analysis for YAP, F-actin, and nuclei in stem cells cultured for 48 hours in a medium without an inhibitor and a medium with actin polymerization inhibitor (cytochalasin D) by using the nanocoil-substrate complex according to the present invention.

FIG. 24 is a result of the confocal immunofluorescent analysis for TAZ, F-actin, and nuclei in stem cells cultured for 48 hours in a medium without an inhibitor and a medium with actin polymerization inhibitor (cytochalasin D), ROCK inhibitor (Y27632), and myosin II inhibitor (blebbistatin) by using the nanocoil-substrate complex according to the present invention.

FIG. 25 is a result of an experiment for host stem cell adhesion control in vivo by using the nanocoil-substrate complex according to the present invention.

FIG. 26 is a graph illustrating adherent cell density, cell area, focal adhesion number, aspect ratio (major axis/minor axis ratio), and nuclear/cytoplasmic YAP fluorescence ratio calculated from the confocal immunofluorescent image of FIG. 25.

### DETAILED DESCRIPTION

Hereinafter, in order to describe the present invention in more specifically, an exemplary embodiment of the present invention will be described in more detail with reference to the accompanying drawings. However, the present invention is not limited to the exemplary embodiment described herein, and may also be specified in other forms.

The present invention provides a nanocoil-substrate complex for controlling adhesion and differentiation of stem cells, the nanocoil-substrate complex including: a substrate; one or more nanocoils chemically coupled to the substrate; and one or more integrin ligand peptides chemically coupled to the nanocoil, in which the nanocoil is provided with a nanowire in a spiral form, includes one or more metal elements, has a length of 100 nm to 20 μm, and has a length reversibly changed depending on application/non-application of a magnetic field within a range of Equation 1 below.

|L₁−L₀|>10 nm [Equation 1]

In Equation 1, L₁ is a length of the nanocoil when a magnetic field is applied, and L₀ is a length of the nanocoil when a magnetic field is not applied.

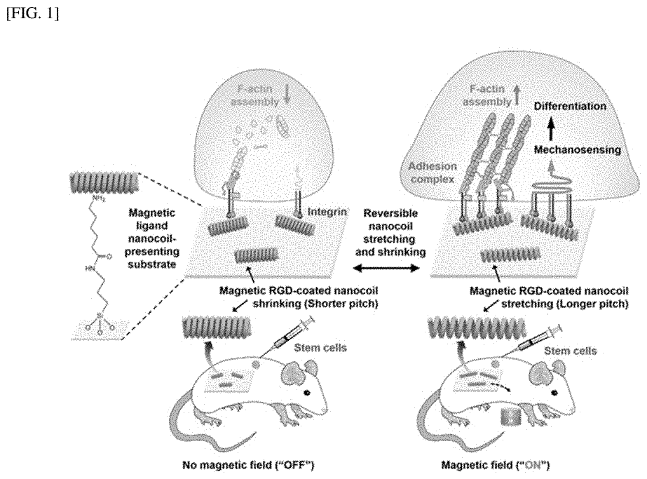

FIG. 1 is a schematic diagram illustrating a nanocoil-substrate complex for controlling cell adhesion and differentiation of stem cells and a method of controlling adhesion and differentiation of stem cells by using the same according to an exemplary embodiment of the present invention.

Referring to FIG. 1, the nanocoil-substrate complex of the present invention includes: a substrate; one or more nanocoils chemically coupled to the substrate; and one or more integrin ligand peptides chemically coupled to the nanocoil, in which the nanocoil is provided with a nanowire twisted in a spiral form and the nanowire includes one or more metal elements among cobalt (Co), iron (Fe), and nickel (Ni).

In particular, the nanocoil may be provided with a nanowire in the spiral form satisfying Equation 1.

|L₁−L₀|>10 nm [Equation 1]

In Equation 1, L₁ is a length of the nanocoil when a magnetic field is applied, and L₀ is a length of the nanocoil when a magnetic field is not applied.

In Equation 1, the length of the coil when the magnetic field is not applied may be 100 nm to 20 μm, 500 nm to 4 μm, or 1 μm to 3 μm.

As described above, when the magnetic field is applied, the nanocoil is stretched and has an increasing length, thereby promoting adhesion of stem cells in vivo. However, when the magnetic field is removed, the nanocoil is compressed, so that the length of the nanocoil returns to the existing length.

In particular, in Equation 1, it can be seen that a change in the length of the nanocoil depending on application/non-application of the magnetic field may be 10 nm or more, 20 nm or more, 10 nm to 500 nm, or 10 nm to 100 nm.

When the change in the length of the nanocoil in the nanocoil-substrate complex of the present invention does not satisfy Equation 1, the change in the length of the nanocoil is small, so there is no difference in cell adhesion, which is a problem.

An average length of a spiral outer diameter of the nanocoil may be 50 nm to 200 nm, or 100 nm to 200 nm. When the spiral outer diameter of the nanocoil is less than 100 nm, the nanocoil is too small, so that it is difficult for the integrin ligand peptide to be coupled at regular intervals, and when the spiral outer diameter of the nanocoil is larger than 200 nm, an area occupied by the nanocoil on the substrate is large, so that there is a problem in that it is difficult to distribute the nanocoils on the substrate at an appropriate density.

The nanocoil is formed of a nanowire, and the nanowire may include one or two or more metal elements among cobalt (Co), iron (Fe), and nickel (Ni), and the nanowire may be provided in the form of a wire having a circular cross-section, and has a diameter of 5 nm to 100 nm, 20 nm to 90 nm, or 60 nm to 90 nm. When the foregoing diameter of the wire is not satisfied, the nanocoils may not exhibit smooth stretching and compression.

The integrin ligand peptide coupled into the nanocoil may be a thiolated integrin ligand peptide, and the plurality of integrin ligand peptides is coupled to the nanocoil while being spaced apart from each other, and an average interval between the adjacent integrin ligand peptides may be 1 nm to 10 nm. When the average interval between the adjacent integrin ligand peptides is less than 1 nm, it is difficult to activate adhesion and differentiation of stem cells even in the case where a magnetic field is applied, and when the average interval between the adjacent integrin ligand peptides is larger than 10 nm, adhesion and differentiation of stem cells are activated even in the case where a magnetic field is not applied, so that there is a problem in that it is difficult to reversibly control the adhesion and differentiation of stem cells by using the magnetic field.

When the magnetic field is applied to the nanocoil, the adjacent spirals in the nanocoil are spaced apart from each other, and a pitch between the adjacent spirals may be 1 nm to 100 nm, 1 nm to 50 nm, or 5 nm to 30 nm. In this case, when the magnetic field is applied, the pitch interval increases while the nanocoil is stretched. Accordingly, an interval between the integrin ligand peptides may also increase.

The integrin ligand peptide is the thiolated integrin ligand peptide, and a thiol group of the integrin ligand peptide may be coupled to the spiral nanocoils by a polyethylene glycol linker. The polyethylene glycol linker may be maleimide-poly(ethylene glycol)-NHS ester (Mal-PEG-NHS ester). The nanocoil includes the polyethylene glycol linker, so that coupling force between the nanocoil and the integrin ligand peptide increases to improve durability.

The nanocoil may have a structure in which carboxylate is coupled. The carboxylate substituent may be an amino acid derivate, in particular, aminocaproic acid. As described above, the nanocoil has the structure in which carboxylate is coupled, thereby increasing coupling force between the nanocoil and the substrate and the integrin ligand peptide.

The substrate is the substrate of which a surface is aminated, and may be the substrate, of which the surface is activated, by soaking the substrate in an aminosilane solution, and may have a structure in which the amino group on the surface of the substrate is coupled to a carboxyl group of the nanocoil through the EDC/NHS reaction.

Further, the substrate may be the substrate which is not coupled with the nanocoil and of which the surface is inactivated.

Further, the present invention provides a method of preparing a nanocoil-substrate complex for controlling adhesion and differentiation of stem cells, the method including: preparing a nanocoil by electrodepositing a solution containing one or more metal elements; coupling a carboxylate substituent to the nanocoil by mixing the nanocoil and a first suspension; manufacturing a substrate coupled with the nanocoil by soaking a substrate of which a surface is activated in a solution containing the nanocoil to which the carboxylate is coupled; coupling a linker to a distal end of the nanocoil by soaking the nanocoil-coupled substrate in a solution containing a polyethylene glycol linker; and coupling an integrin ligand peptide to the nanocoil by mixing a second suspension containing the integrin ligand peptide (RGD) and the activated substrate coupled with the nanocoil.

In the preparing of the nanocoil, the solution containing the metal element may include one or two or more metal elements among cobalt (Co), iron (Fe), and nickel (Ni).

The preparing of the nanocoil includes: preparing a nano template including nano pores, and including a working electrode on one surface thereof; preparing a first metal precursor mixed solution containing a metal precursor solution containing ascorbic acid (C₆H₈O₆), vanadium (IV) oxide sulfate (VOSO₄.xH₂O), and a metal to be deposited; preparing a second metal precursor mixed solution by mixing the first metal precursor mixed solution and nitric acid (HNO₃); immersing the nano template in the second metal precursor mixed solution, and depositing metal nanocoils on the nano pores by an electrodepositing method by applying a current between a counter electrode and the working electrode inserted into the second metal precursor mixed solution; and selectively removing the working electrode and the nano template in the nano template on which the metal nanocoils are deposited.

As the nano template, an Anodic Aluminum Oxide (AAO) nanoframe, an inorganic nanoframe, or a polymer nanoframe is used. Herein, the case of using the AAO nanoframe is illustrated. A size of the nanowire is determined according to a diameter of a pore of the AAO nanoframe, and a length of the nanowire is determined according to a forming time and speed of the nanowire.

An average diameter of the nano pore may be 5 to 500 nm, 50 nm to 200 nm, or 100 nm to 200 nm.

The metal precursor solution may include at least one of cobalt sulfate (II) heptahydrate (CoSO₄.7H₂O) and iron sulfate (II) heptahydrate (FeSO₄.7H₂O).

A concentration of cobalt sulfate (II) heptahydrate (CoSO₄.7H₂O) may be 30 mM to 100 mM, a concentration of vanadium(IV) oxide sulfate (VOSO₄.xH₂O) may be 30 mM to 100 mM, a concentration of iron sulfate(II) heptahydrate (FeSO₄.7H₂O) may be 30 mM to 100 mM, and a concentration of ascorbic acid (C₆H₈O₆) may be 20 mM to 50 mM.

pH of the second mixed precursor mixed solution may be 1.5 to 2.5.

The method may further include immersing the nano template in the second metal precursor mixed solution and decompressing a plating bath containing the second metal precursor mixed solution. Pressure of the plating bath may be 100 Torr to 700 Torr.

A density of a current flowing in the working electrode during the electroplating may be 0.1 to 300 mA/cm², and an electroplating time may be one minute to 48 hours.

A silver (Ag) electrode layer having a thickness of 250 nm is formed on a bottom surface of the AAO nanoframe by an electron beam evaporation method. The electrode layer serves as a negative electrode during the electrodeposition. Herein, as the electrode layer, other metals or other conductive material layers may be used.

The coupling of the carboxylate substituent may be performed by mixing the nanocoil and the first suspension and reacting the nanocoil and the first suspension for 8 to 20 hours to 10 to 15 hours. The first suspension may contain an amino acid derivative containing a carboxylate substituent, and specifically, the amino acid derivative may be aminocaproic acid. The amino acid derivative may be coupled to the surface of the nanocoil by reacting the nanocoil with the first suspension.

The manufacturing of the substrate coupled with the nanocoil may be performed by soaking the substrate, of which the surface is activated, in the solution containing the nanocoil in which the carboxylate is couple.

The substrate, of which the surface is activated, may be manufactured by immersing the substrate in the acidic solution containing any one or more of hydrochloric acid and sulfuric acid for 30 minutes to 2 hours or 30 minutes to 1 hour. Through this, the coupling with an amino group is facilitated by coupling a hydroxyl group to the surface of the substrate, thereby effectively performing activation of the surface of the substrate.

In the manufacturing of the substrate coupled with the nanocoil, the surface of the substrate may be aminated by soaking the substrate, of which the surface is activated, in the amino-silane solution under a dark condition. The amino-silane solution may include (3-aminopropyl)triephoxysilane (APTES). In this case, the amination of the surface of the substrate means that the amine group is coupled onto the substrate. The surface of the substrate is aminated by immersing the substrate in the amino-silane solution, so that the substrate may be coupled with the nanocoil through the EDC/NHS reaction.

The coupling of the linker to the distal end of the nanocoil may be performed by soaking the nanocoil-coupled substrate in the solution containing the polyethylene glycol linker. The polyethylene glycol linker may be maleimide-poly(ethylene glycol)-NHS ester (Mal-PEG-NHS ester). The nanocoil includes the polyethylene glycol linker, so that coupling force between the nanocoil and the integrin ligand peptide increases to improve durability.

The coupling of the integrin ligand peptide to the nanocoil may be performed by mixing a second suspension including the integrin ligand peptide (RGD) and the activated nanocoil-coupled substrate. The second suspension may include the thiolated integrin ligand peptide.

The method may further include soaking the nanocoil-coupled substrate in a solution including a polyethylene glycol derivative and inactivating the surface of the substrate that is not coupled with the nanocoil, after the coupling of the integrin ligand peptide to of the nanocoil. The polyethylene glycol derivative may be methoxy-poly(ethylene glycol)-succinimidylcarboxymethyl ester.

Further, the present invention provides a method of controlling adhesion and differentiation of stem cells, the method including controlling cell adhesion and differentiation of stem cells by treating the nanocoil-substrate complex for controlling cell adhesion and differentiation of the stem cells with a culture medium and then applying a magnetic field in a range from 20 mT to 7 T, and in which a length of the nanocoil is reversibly changed depending on application/non-application of a magnetic field, and the length of the nanocoil satisfies Equation 1 below.

|L₁−L₀|>10 nm [Equation 1]

In Equation 1, L₁ is a length of the nanocoil when a magnetic field is applied, and L₀ is a length of the nanocoil when a magnetic field is not applied.

In the controlling of the cell adhesion and the differentiation of the stem cells, it is possible to control adhesion and differentiation of stem cells in vivo and ex vivo by reversibly changing the length of the nanocoil depending on application/non-application of the magnetic field to the nanocoil-substrate complex.

In particular, in the controlling of the adhesion and the differentiation of the stem cells, when the magnetic field is not applied to the nanocoil-substrate complex, the nanocoil is compressed and a pitch interval of the nanocoil is decreased to degrade adhesion and mechanosensing differentiation of stem cells.

Further, in the controlling of the cell adhesion and the differentiation of the stem cells, when the magnetic field is applied to the nanocoil-substrate complex, the nanocoil is stretched and a pitch interval of the nanocoil is increased to promote adhesion and mechanosensing differentiation of stem cells.

For example, when the magnetic field is applied to the nanocoil-substrate complex and then the magnetic field is removed, the nanocoil is reversibly stretched and compressed. In particular, when the magnetic field is applied to the nanocoil-substrate complex, the magnetic field is removed, and then the magnetic field is applied to the nanocoil-substrate complex again, the nanocoil may be stretched, compressed, and then stretched again.

Accordingly, it is possible to temporally and reversibly control the cell adhesion and differentiation of stem cells by using the nanocoil-substrate complex according to the present invention.

In particular, in Equation 1, the change in the length of the nanocoil depending on application/non-application of the magnetic field may be 10 nm or more, 20 nm or more, 10 nm to 500 nm, or 10 nm to 100 nm.

When the change in the length of the nanocoil in the nanocoil-substrate complex of the present invention does not satisfy Equation 1, the change in the length of the nanocoil is small, so there is no difference in cell adhesion performance, which is a problem.

Hereinafter, examples of the present invention will be described. However, the examples below are merely preferable examples of the present invention, and the scope of the present invention is not limited by the examples.

### PREPARATION EXAMPLE

### Preparation Example

Prepare Nanocoil

A nanocoil was prepared by using an AAO porous template having pores with 200 nm in diameter through electrodeposition. First, silver (Ag) was deposited on one surface of the AAO porous template by using an electronbeam evaporator. A metal ion precursor solution was prepared by mixing cobalt sulfate heptahydrate (CoSO₄.7H₂O, 0.08M) and iron sulfate heptahydrate (FeSO₄.7H₂O, 0.08M) in deionized water. In order to produce CoFe nanocoils, vanadium (IV) oxide sulfate (VOSO₄.xH₂O), and L-ascorbic acid (0.06 M) were added to the metal ion precursor solution. Next, nitric acid was then added to the precursor solution to adjust the pH to 2.5, the mixed precursor solution was injected into the pores of the AAO template pores, and then a current at constant current density of 20 mA/cm² was applied to deposit CoFe nanocoils. The nanotemplate was removed by reacting the CoFe nanocoil-deposited nanotemplate and 1 M of NaOH for 30 minutes at 45° C., followed by washing the CoFe nanocoil with deionized water to prepare the CoFe nanocoils. The washed CoFe nanocoils were suspended in 1 mL of deionized water before being coupled to the substrate.

### Comparative Preparation Example

A nanocoil was prepared by the same method as that of Preparation Example 1 except that a negatively charged thiolated RGD peptide (CDDRGD, GL Biochem) was not added.

### Example

### Example

Prepare Nanocoil-Substrate Complex

Aminocaproic acid was used to be coupled to a surface of a magnetic CoFe nanocoils based on an amine group that is reported to react with the native oxide layer of the nanocoils prepared in the preparation example. A mixed solution of 1 mL of nanocoils and 1 mL of 6 mM of an aminocaproic acid solution were stirred at a room temperature for 12 hours, and then centrifuged and washed with deionized water. A cell culture grade glass substrate (22 mm×22 mm) was aminated to allow a carboxylate group on the surface of the nanocoil to be bonded to the amine group on the substrate. The substrate was first washed with a mixture in which hydrochloric acid and methanol were mixed at a ratio of 1:1 for 30 minutes and rinsed with deionized water. The washed substrate was activated in sulfuric acid for 1 hour and washed with DI water. The substrate was aminated in 3-aminopropyl triethoxy silane (APTES) and ethanol (1:1) in a darkroom for 1 hour and washed with ethanol, followed by drying for 1 hour at 100° C. The aminocaproic acid-conjugated nanocoils were activated in 1 mL of deionized water containing 0.5 mL of 20 mM N-ethyl-N′-(3-(dimethylaminopropyl)carbodiimide) (EDC) and 0.5 mL of 20 mM N-hydroxysuccinimide (NHS) through EDC/NHS reaction for 3 hours, followed by washing with deionized water.

The aminated substrate was incubated with the activated nanocoils for 1 hour, followed by washing with deionized water. An integrin ligand was cultured in 1 mL of deionized water containing 0.04 mM of maleimide-poly(ethylene glycol)-NHS linker and 2 μl of N,N-Diisopropylethylamine (DIPEA) under the shaking in the dark for 16 hours, grafted on the surface of the substrate by mediating the amide bond formation, followed by washing with deionized water. To mediate the thiol-ene reaction, the substrate was cultured in 1 mL of deionized water containing thiolated RGD peptide ligands (GCGYGRGDSPG, GL Biochem, 0.04 M), 2 μL of N,N-diisopropylethylamine (DIPEA), and 10 mM Tris(2-carboxyethyl)phosphine hydrochloride (TCEP) for 2 hours in the dark, and then washed with deionized water. To minimize the non-RGD ligand specific stem cell adhesion before the culturing of the cell, the areas to which the nanocoil was not coupled were activated in 1 mL of deionized water containing 2 μL of N,N-diisopropylethylamine (DIPEA) and 100 μM methoxy-poly(ethylene glycol)-succinimidyl carboxymethyl ester in the dark for 2 hours, followed by washing to block the non-nanocoil-coated area of the substrate.

### Comparative Example 1

A nanocoil-substrate complex was prepared by the same method except for using the prepared nanocoil Comparative Preparation Example 1.

### Experimental Example

### Experimental Example 1

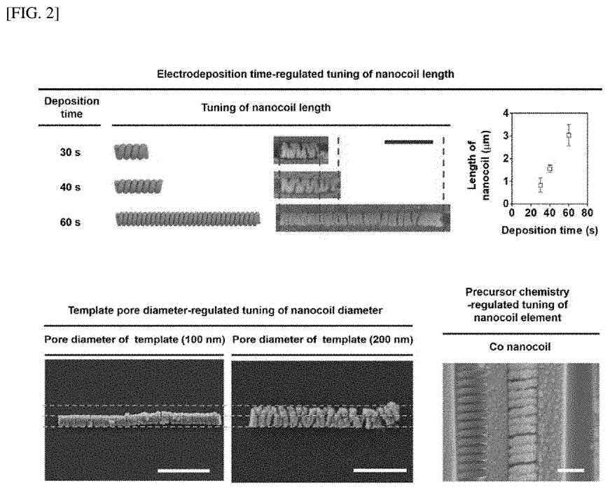

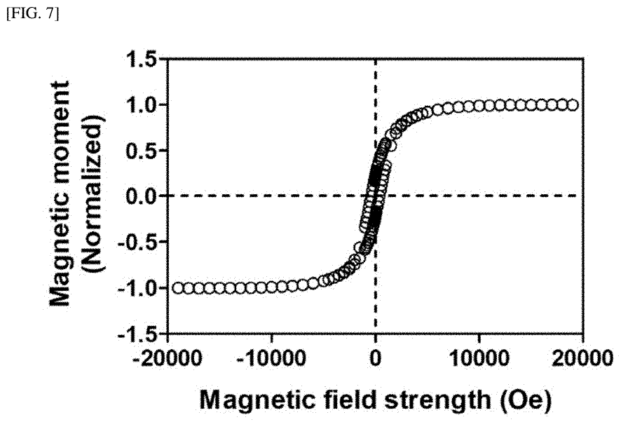

In order to check the form and the chemical characteristic of the nanocoil according to the present invention, the prepared nanocoils were photographed by using a Scanning Electron Microscope (SEM), a High-Angle Annular Dark-Field Scanning Transmission Electron Microscopy (HAADF-STEM), a High-Resolution Transmission Electron Microscope (HR-TEM), and a High-Resolution Scanning Transmission Electron Microscopy (HR-STEM), and then analyzed by using Energy Dispersive X-ray Spectroscopy (EDS), Electron Energy Loss Spectroscopy (EELS), Vibrating-Sample Magnetometry (VSM), and X-ray Diffraction (XRD), and a result thereof is represented in FIGS. 2 and 7.

FIG. 2 is an SEM image of the nanocoil according to the present invention. In particular, an upper part of FIG. 2 is an SEM image and a graph of the measured length of the CoFe nanocoil according to an electrodeposition time, a lower left part of FIG. 2 is an SEM image of the CoFe nanocoil prepared by adjusting a pore diameter of the electrodeposition template, and a lower right part of FIG. 2 is an SEM image of the cobalt nanocoil and the CoFe nanocoil, and in this case, a scale bar represents 1 μm, 500 nm, and 200 nm respectively.

Referring to FIG. 2, according to the nanocoil according to the present invention, it can be seen that it is possible to regulate the diameter of the CoFe nanocoil according to the pore diameter of the electrodeposition template, it is possible to control a constituent element of the nanocoil according to the control of the metal ion precursor, and it is possible to regulate the length of the CoFe nanocoil according to an electrodeposition time.

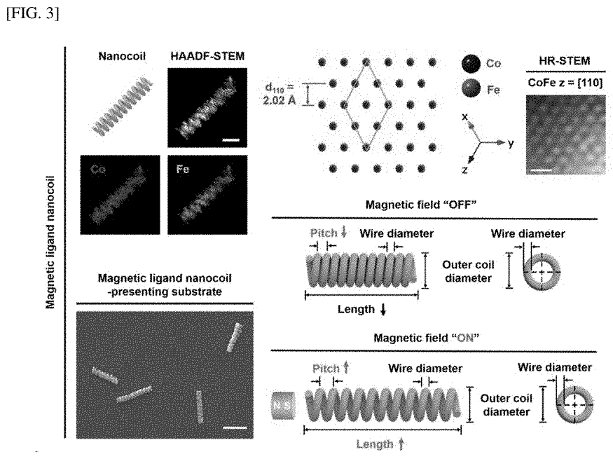

FIG. 3 is a High-Angle Annular Dark Field Scanning Transmission Electron Microscope (HAADF-STEM) image, and an Energy Dispersive Spectroscopy (EDS) mapping image, and a High-Resolution Scanning Transmission Electron Microscopy (HR-STEM) image of the nanocoil according to the present invention.

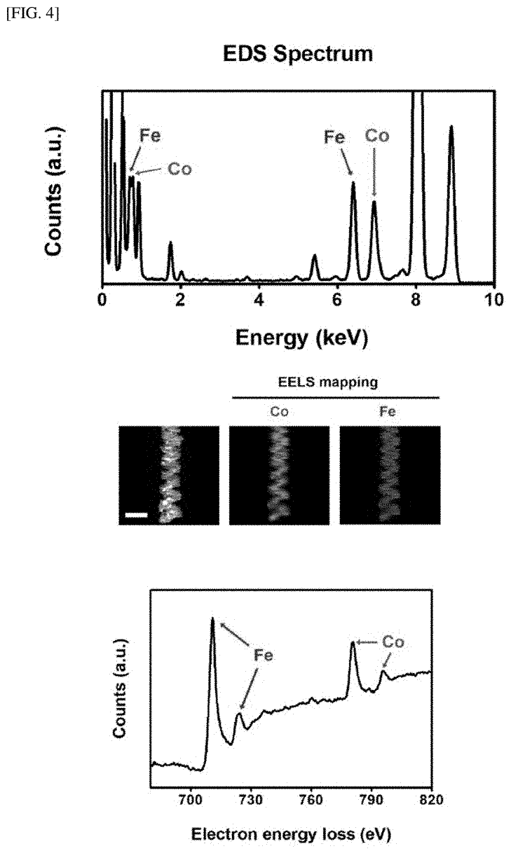

FIG. 4 is a graph illustrating an EDS analysis result, and a graph and a mapping image illustrating an EELS analysis result of the nanocoil according to the present invention.

Referring to FIGS. 3 and 4, in the HAADF-STEM and EELS maaping image, it can be seen that the nanocoil consists of cobalt (Co) and iron (Fe), each of which is constantly distributed with a distribution of about 50 atom %.

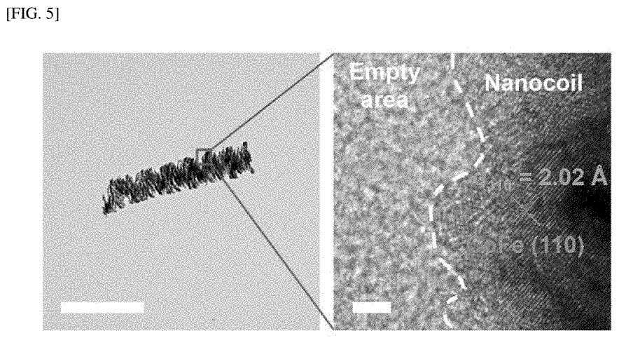

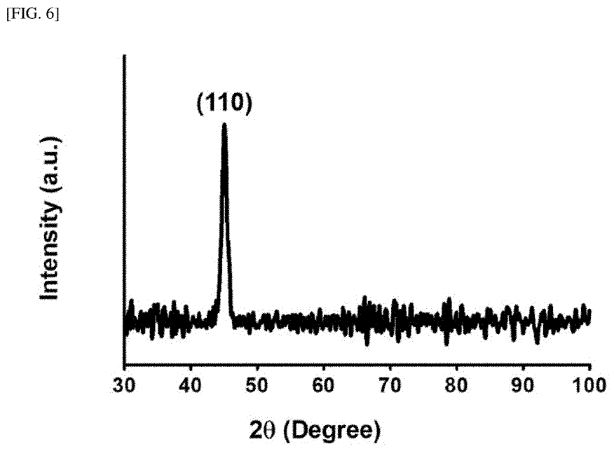

FIG. 5 is a High-Resolution Transmission Electron Microscopy (HRTEM) image of the nanocoil according to the present invention, and FIG. 6 is an X-ray diffraction analysis graph of the nanocoil according to the present invention.

Referring to FIGS. 5 and 6, it can be seen that the nanocoil has a (110) crystal plane of a body centered cubit structure, and has an average lattice interval of about 2.02±0.02 Å. Further, it can be seen that in order to promote the coupling with the isotropic integrin ligand, the diameter of the nanowire forming the nanocoil is almost similar to about 10 nm that is an integrin molecule size.

FIG. 7 is a graph illustrating a vibrating-sample magnetometry measurement result of the nanocoil according to the present invention. In particular, the magnetic characteristic of the nanocoil by cobalt and iron was confirmed, and through this, it can be recognized that reversible bimodal switching between nano stretching (“ON”) and nano-compression (“OFF) of the nanocoil is possible.

### Experimental Example 2

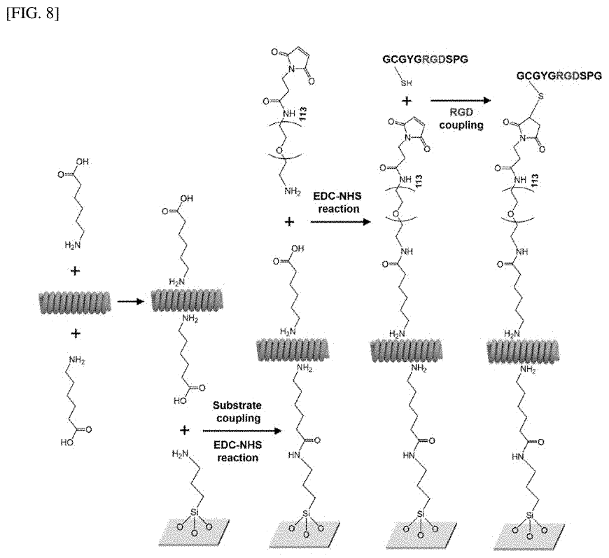

In order to confirm the characteristic of the nanocoil-substrate complex according to the present invention, the nanocoil-substrate complex was photographed with a Field Emission Scanning Electron Microscope (FE-SEM), the Fourier-Transform Infrared Spectroscopy (FT-IR) was carried, and the nanocoil-substrate complex was photographed with an Atomic Force Microscope (AFM), and the results thereof are represented in FIGS. 8 to 11.

The FT-IR was conducted by using GX1 (Perkin Elmer Spectrum, USA) in order to confirm the chemical bond characteristics of the nanocoils. The samples analyzed for the change in chemical bond characteristics were lyophilized and densely packed into KBr pellet prior to the analysis.

FIG. 8 is an image schematically illustrating an operation of preparing a nanocoil-substrate complex according to the present invention. Referring to FIG. 8, aminocaproic acid was coupled to the nanocoil. Next, the aminocaproic acid-bonded nanocoil was put in water containing EDC and NHS and activated by using the EDC/NHS reaction, and then was coupled to the substrate of which the surface is aminated. Polyethylene glycol was coupled to aminocaproic acid coupled to the nanocoil that is not coupled with the substrate, and the integrin ligand was coupled to the nanocoil by reacting the polyethylene glycol and the thiolated integrin ligand (RGD).

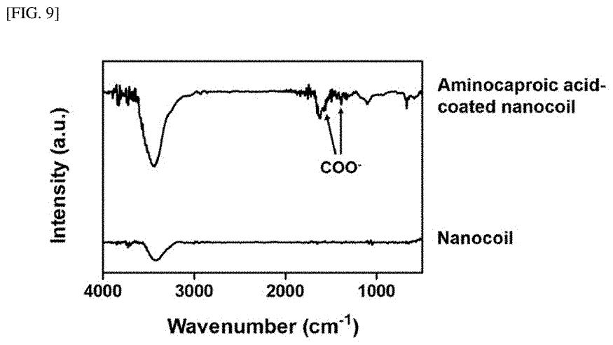

FIG. 9 is a diagram illustrating a result of a Fourier Transform Infrared Spectroscopy (FT-IR) analysis of the nanocoil-substrate complex according to the present invention. Referring to FIG. 9, the chemical bond characteristics of the aminocaproic acid-coated nanocoil can be recognized. In particular, COO⁻ binding was confirmed at 1560-1565 cm⁻¹ and 1387-1389 cm⁻¹. Through this, it can be seen that aminocaproic acid was successfully coupled to the nanocoil.

In addition, in order to minimize adhesion of non-ligand-specific stem cells, the substrate that is not coupled with the nanocoil was coupled with a methoxy-PEG-NHS ester group to be inactivated, and referring to FIG. 3, the uniform distribution of the nanocoils can be confirmed through the scanning electron microscope, and it can be seen that a density of the nanocoils is about 62802±2385 nanocoils/mm².

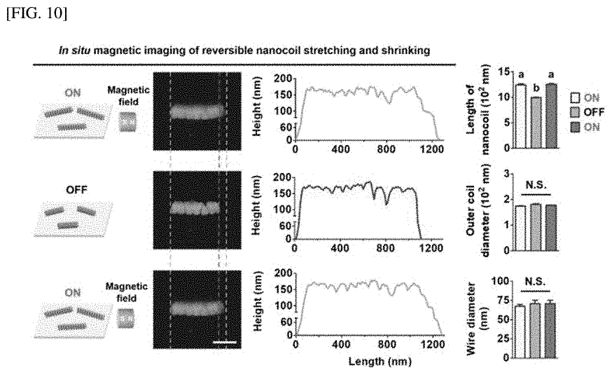

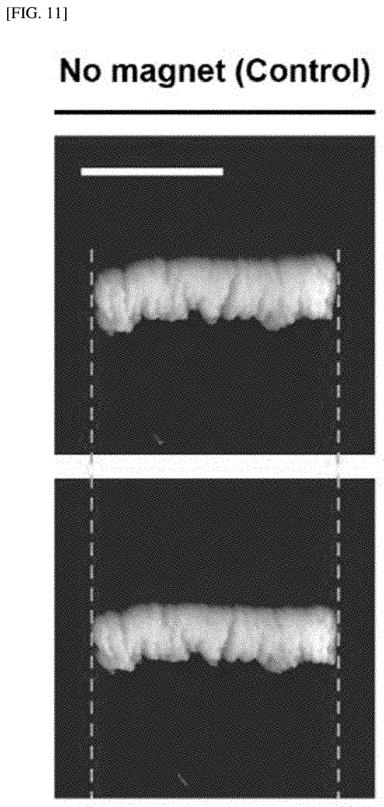

FIG. 10 is a diagram illustrating the result obtained by using the AFM in order to confirm magnetic bimodal switching of an elastic motion with stretching (“ON”) and compression (“OFF”) of the nanocoil according to the present invention. FIG. 11 is a diagram illustrating the result obtained by photographing the case where a magnetic field is not applied to the nanocoil according to the present invention by using the AFM.

Referring to FIGS. 10 and 11, it can be seen that in the nanocoil-substrate complex according to the present invention, when a magnetic field is applied, the nanocoil is stretched, so that the length of the nanocoil increases, and when the magnetic field is not applied again, the nanocoil is compressed, so that the length of the nanocoil returns to the original state. However, it can be seen that only the length of the nanocoil is simply increased and then decreased again, but the outer diameter of the nanocoil or the diameter of the nanowire forming the nanocoil is not significantly different.

In particular, it can be seen that the length of the nanocoil when the magnetic field is applied is 1243±28 nm, when the magnetic field is not applied, the length of the nanocoil is decreased to 995±4 nm, and when the magnetic field is applied again, the length of the nanocoil is increased to 1255±18 nm. In this case, the diameter of the nanocoil is maintained with 174 nm to 180 nm, and the diameter of the nanowire forming the nanocoil is maintained with 66 to 71 nm, so that it can be seen that the outer diameter and the wire diameter of the nanocoil remained similar without significant differences during the cyclic switching “OFF”, “ON”, and “OFF”.

Through this, it can be seen that in the nanocoil-substrate complex of the present invention, the macroscale ligand density is constantly maintained during the bimodal switching.

### Experimental Example 3

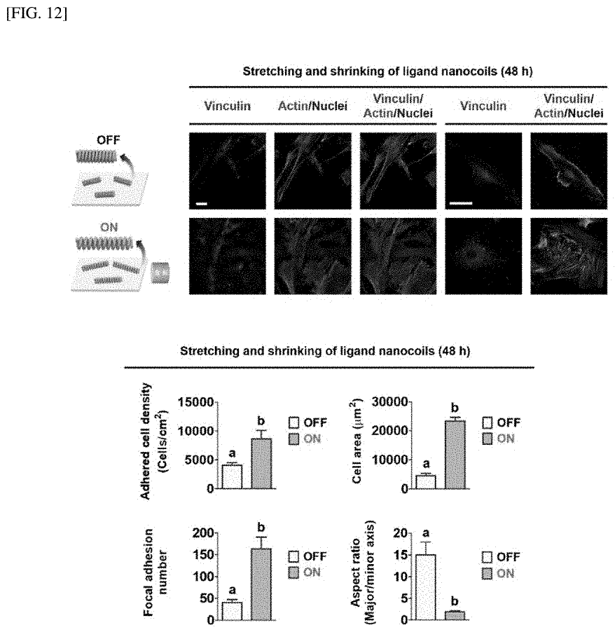

The following experiment was conducted to confirm an influence on the adhesion of stem cells according to the application of a magnetic field to the nanocoil-substrate complex according to the present invention, and the result thereof is represented in FIGS. 12 to 16.

To investigate the influence of magnetic switching of reversible strength and compression of the ligand-containing nanocoil on focal adhesion, mechanosensing and differentiation of the stem cells, the evaluation was conducted by using the nanocoil-substrate complex prepared in the preparation example. The substrate was sterilized under ultraviolet light for 2 hours prior to the use of the substrate. Human mesenchymal stem cells (hMSCs, passage 5 from Lonza) were plated on the sterilized substrate at a density of approximately 9,500 cells/cm² and cultured in growth medium containing high glucose Dulbecco's Modified Eagle Medium (DMEM) supplemented with 10% fetal bovine serum, 4 mM L-glutamine, and 50 U/mL penicillin/streptomycin at 37° C. under 5% CO₂.

The focal adhesion and mechanosensing of the stem cells was investigated by placing a permanent magnet (270 mT) near the edge of the materials (“ON”) for 48 hours to promote the in situ stretching of the nanocoils toward the edge of the materials or removing the magnet (“OFF”) for 48 hours to induce reversible compression of the nanocoils to the original structures. The control experiment to evaluate the focal adhesion and mechanosensing of the stem cells was performed under bimodal switching (application/removal of the magnetic field), but was performed in the state where there was no nanocoil or integrin ligand.

FIG. 12 is a confocal immunofluorescent image of F-actin, nuclei, and vinculin in stem cells cultured (after 48 hours) by using the nanocoil-substrate complex according to the present invention (an upper part), and a graph illustrating an adherent cell density, a cell area, focus adherence number, and an aspect ratio (a ratio of major axis/minor axis) calculated based on the result of the confocal immunofluorescent experiment (a lower part), and a scale bar represents 50 μm.

Referring to FIG. 12, it can be seen that the stretching (“ON”) mode in which the magnetic field is applied exhibits considerably higher adhesive cell density and focal adhesion throughout the wider area than the compression (“OFF”) mode in which the magnetic field is removed, and promotes vinculin clustering in the focal adhesion complex.

The effect of cyclic switching “ON” (stretching) and “OFF” (compression) on the focal adhesion of stem cells was investigated under the placement (“ON”) or removal (“OFF”) of the magnet for 54 hours or the cyclic switching every 18 hours (“OFF-OFF-OFF”, “OFF-ON-OFF”, “ON-OFF-ON”, and “ON-ON-ON” groups).

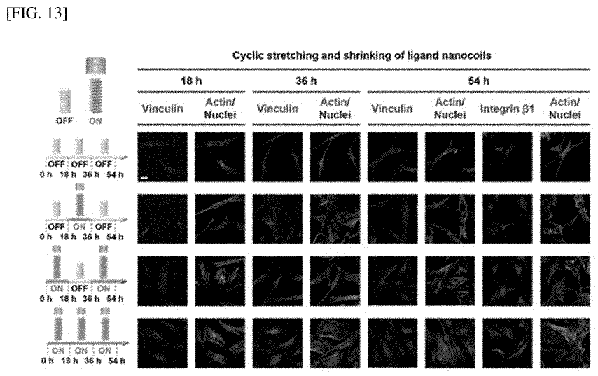

FIG. 13 is a confocal immunofluorescent image of F-actin, nuclei, and vinculin in stem cells cultured (after 54 hours) by changing the application of a magnetic field every 18 hours by using the nanocoil-substrate complex according to the present invention, and a scale bar represents 50 μm.

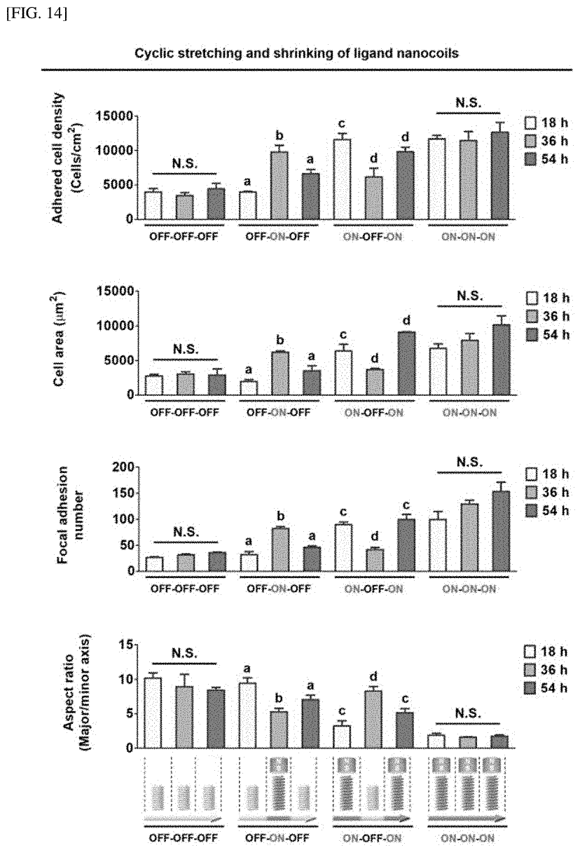

FIG. 14 is a graph illustrating an adherent cell density, a cell area, focal adherence number, and an aspect ratio (a ratio of major axis/minor axis) of the stem cells cultured by changing the application of a magnetic field every 18 hours by using the nanocoil-substrate complex according to the present invention calculated based on the result of the confocal immunofluorescent experiment.

Referring to FIGS. 13 and 14, “ON” (stretching) and “OFF” (compression) that are the bimodal switching of the nanocoil-substrate complex promote and suppress reversible integrin β1 expression and focal adhesion of stem cells in the repeated cycle, respectively. In particular, it can be seen that in the stretching mode in which the magnetic field is applied, the focal adhesion of the stem cells is promoted, and in the compression mode in which the magnetic field is removed, the focal adhesion of the stem cells is suppressed. Accordingly, it can be seen that in the case where the magnetic field is applied and then removed, adherent cell density, cell area, and focal adhesion number are increased and then decreased.

To confirm the cytocompatibility of the stretching and compression of CoFe nanocoils on adherent stem cells, cell staining was performed. The adherent stem cells on the ligand-coated CoFe nanocoils cultured under stretching (“ON”) or compression (“OFF”) conditions at 48 hour were washed with phosphate-buffered saline (PBS) and incubated in a staining solution containing 0.05% green fluorescent calcein-AM and 0.2% red-fluorescent propidium iodide (PI) in DMEM at 37° C. for 30 minutes. The stained cells were washed with PBS and imaged under a fluorescence microscope. The live (green) and dead (red) cells were counted to determine viability of the stem cells.

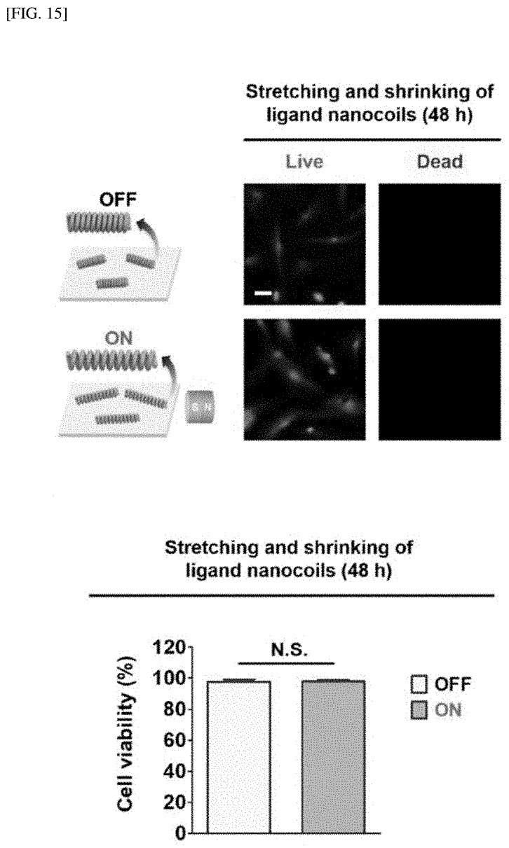

FIG. 15 is a confocal immunofluorescent image of live cells and dead cells in stem cells cultured (after 48 hours) by using the nanocoil-substrate complex according to the present invention (an upper part), and a graph illustrating cell viability calculated based on the result of the confocal immunofluorescent experiment (a lower part), and a scale bar represents 50 μm.

Referring to FIG. 15, it can be seen that cell viability is excellent at 95% in both the stretching mode in which the magnetic field is applied and the compression mode in which the magnetic field is not applied the CoFe nanocoils-substrate complex, so that the CoFe nanocoils-substrate complex does not have no cytotoxicity to stem cells, so that the cytocompatibility is excellent.

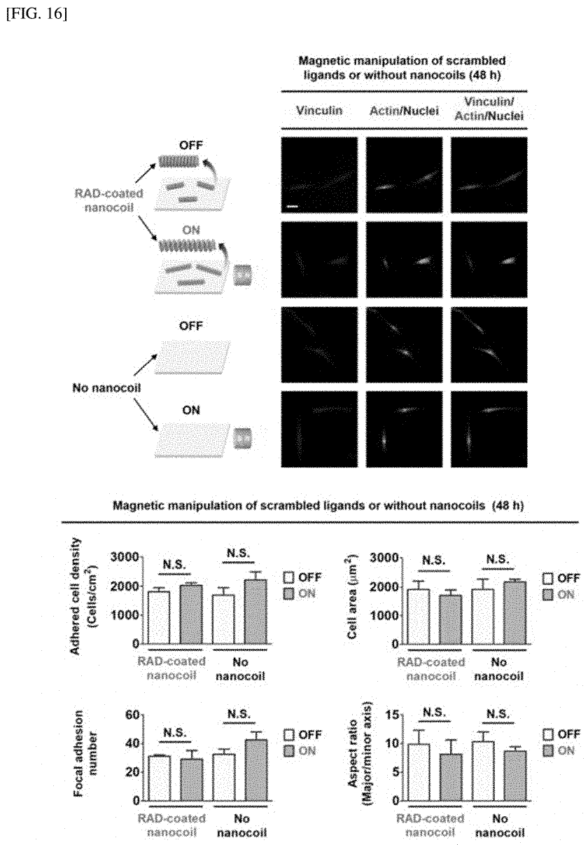

FIG. 16 is a diagram illustrating a result of an experiment for adhesion of stem cells for bimodal switching in a substrate having no nanocoil or the nanocoil-substrate complex to which the integrin ligand (RGD) is not coupled according to a comparative example of the present invention, and an upper part of FIG. 16 is a confocal immunofluorescent image of F-actin, nuclei, and vinculin in stem cells cultured for 24 hours, and a lower part of FIG. 16 is a graph representing an adherent cell density, a cell area, and focal adherence number, and an aspect ratio (a ratio of major axis/minor axis) calculated based on the confocal immunofluorescent experiment result, and in this case, a scale bar represents 50 μm.

Referring to FIG. 14, in the comparative example, there is no significant difference in the bimodal switching “ON” and “OFF” in the state of using the substrate having no nanocoil or the substrate to which the integrin ligand (RGD) is not coupled, so that the adhesion of stem cells is not promoted.

Through this, in the case of the nanocoil-substrate complex of the present invention, the bimodal switching exhibits an effect only when the integrin ligand peptide is coupled to the nanocoil, so that it can be seen that in order to promote and remotely control the stem cell adhesion, both the integrin ligand peptide and the nanocoil are required.

### Experimental Example 4

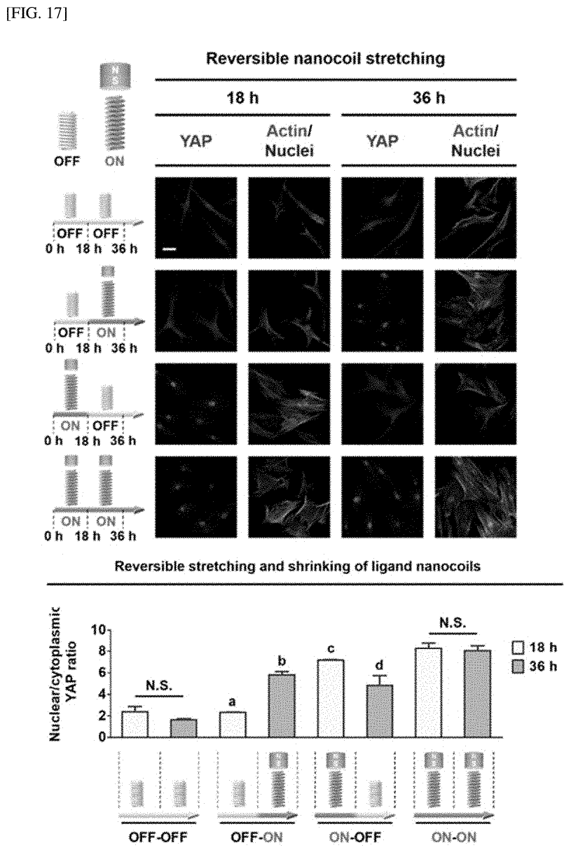

An experiment to find the effect of “ON” (stretching) and “OFF” (compression) that are the remote and reversible bimodal switching on osteogenic differentiation of stem cells by using the nanocoil-substrate complex according to the present invention was conducted as described below, and a result of the experiment is represented in FIGS. 17 to 24.

The integrin ligation-mediated focal adhesion and spreading of stem cells activate mechanotransduction signaling that mediates stem cell differentiation. Cyclic macroscale stretching of cell-adhesive fibronetin and laminin activates the phosphorylation of focal adhesion kinase (FAK) in stem cells to promote their osteogenic differentiation.

FIG. 17 is a confocal immunofluorescent image of F-actin, nuclei, and vinculin in stem cells cultured for 36 hours by adjusting an application of a magnetic field at an interval of 18 hours by using the nanocoil-substrate complex according to the present invention (an upper part), and a graph illustrating nuclei/cytoplasm YAP ratio calculated based on the result of the confocal immunofluorescent experiment (a lower part), and a scale bar represents 50 μm.

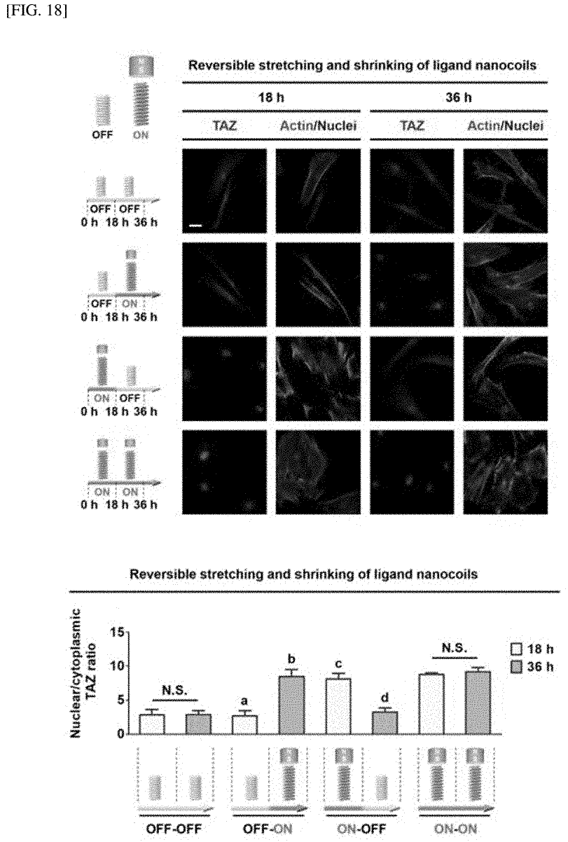

FIG. 18 is a confocal immunofluorescent image of F-actin, nuclei, and TAZ in stem cells cultured for 36 hours by adjusting an application of a magnetic field at an interval of 18 hours by using the nanocoil-substrate complex according to the present invention (an upper part), and a graph illustrating nuclei/cytoplasm YAP ratio calculated based on the result of the confocal immunofluorescent experiment (a lower part), and in this case, a scale bar represents 50 μm.

Referring to FIGS. 17 and 18, it can be confirmed that in the time-regulated bimodal switching, the stretching “ON” mode in which the magnetic field is applied stimulates significantly higher nuclear translocation of YAP/TAZ mechanotransducers of stem cells via immunofluorescence in a reversible manner

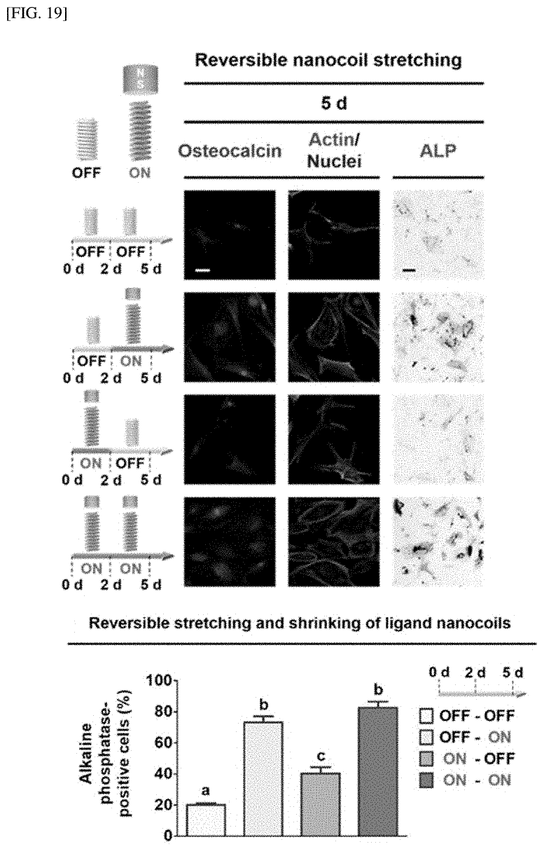

FIG. 19 is a confocal immunofluorescent image and ALP staining image of osteocalcin, F-actin, nuclei in stem cells cultured 5 days by using the nanocoil-substrate complex according to the present invention, in which an application of a magnetic field is adjusted at the second day (an upper part), and a graph representing alkaline phosphatase-positive cell ratio calculated based on the result of the confocal immunofluorescent experiment (a lower part), and in this case, a scale bar represents 50 μm.

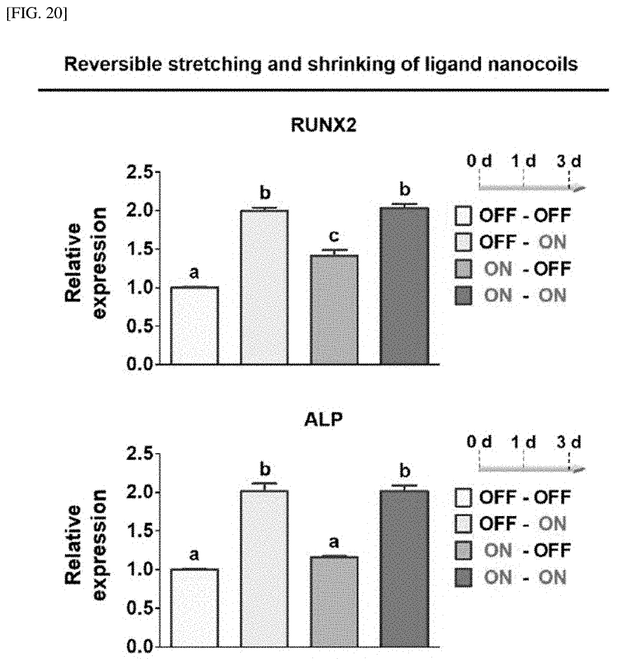

FIG. 20 is a graph illustrating a quantitative analysis of the nuclear/cytoplasmic RUNX2 and ALP gene expression profile in stem cells cultured for 3 days by using the nanocoil-substrate complex according to the present invention, in which an application of a magnetic field is adjusted after one day.

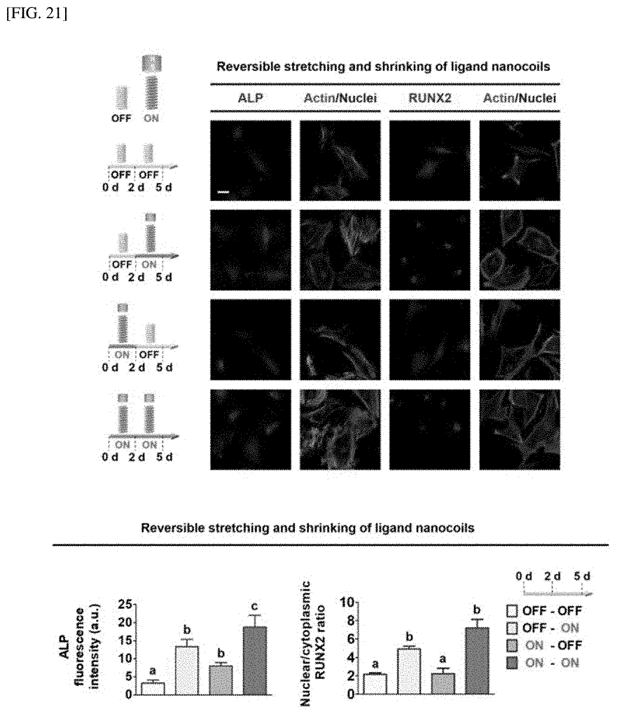

FIG. 21 is a confocal immunofluorescent image of ALP genes, RUNX2, F-actin, and nuclei in stem cells cultured 5 days by using the nanocoil-substrate complex according to the present invention, in which an application of a magnetic field is adjusted at the second day (an upper part), and a graph representing ALP fluorescent intensity and nuclear/cytoplasmic YAP ratio calculated based on the result of the confocal immunofluorescent experiment (a lower part), and in this case, a scale bar represents 50 μm.

Referring to FIGS. 20 and 21, in the time regulated bimodal switching, the stretching (“ON”) in which the magnetic field is applied reversibly facilitates pronounced expression of early markers (significantly higher nuclear translocation in RUNX2, alkaline phosphatase-positive cells, and RUNX2/ALP gene expression) and late marker (pronounced osteocalcin expression) for osteogenic differentiation in stem cells.

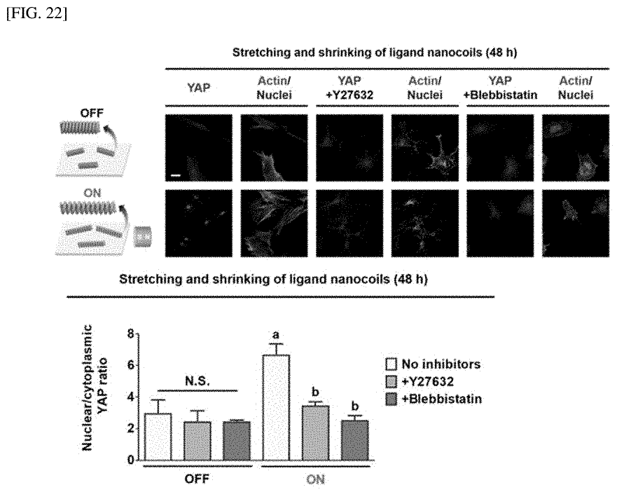

FIG. 22 is a confocal immunofluorescent image of YAP, F-actin, and nuclei in stem cells cultured for 48 hours in a medium without an inhibitor and a medium with ROCK inhibitor (Y27632) and myosin II inhibitor (blebbistatin) by using the nanocoil-substrate complex according to the present invention, and a scale bar represents 50 μm (an upper part), and is a graph representing a result of a calculation of nuclear/cytoplasmic YAP fluorescence ratio by Y27632 and blebbistatin calculated from the confocal immunofluorescent image (a lower part).

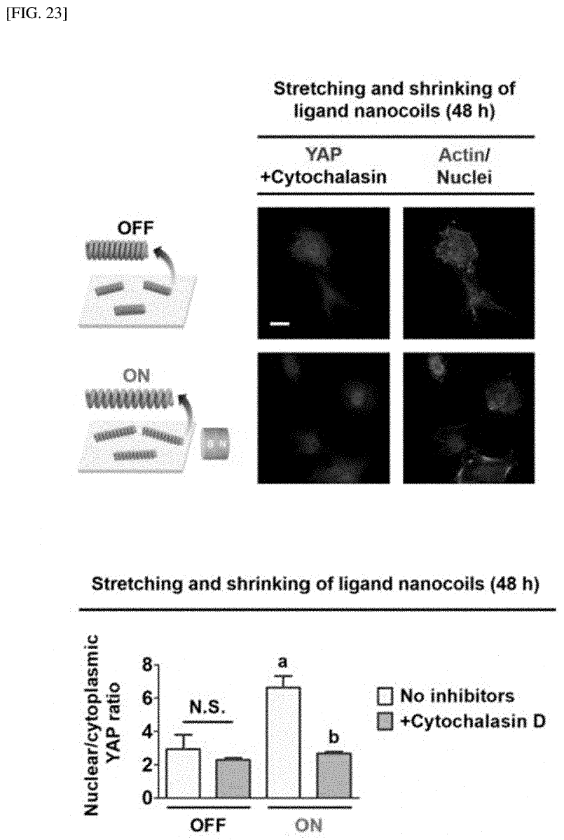

FIG. 23 is a confocal immunofluorescent image of YAP, F-actin, and nuclei in stem cells cultured for 48 hours in a medium without an inhibitor and a medium with actin polymerization inhibitor (cytochalasin D) by using the nanocoil-substrate complex according to the present invention, and a scale bar represents 50 μm (an upper part), and is a graph representing a result of a calculation of nuclear/cytoplasmic YAP fluorescence ratio by cytochalasin D calculated from the confocal immunofluorescent image (a lower part).

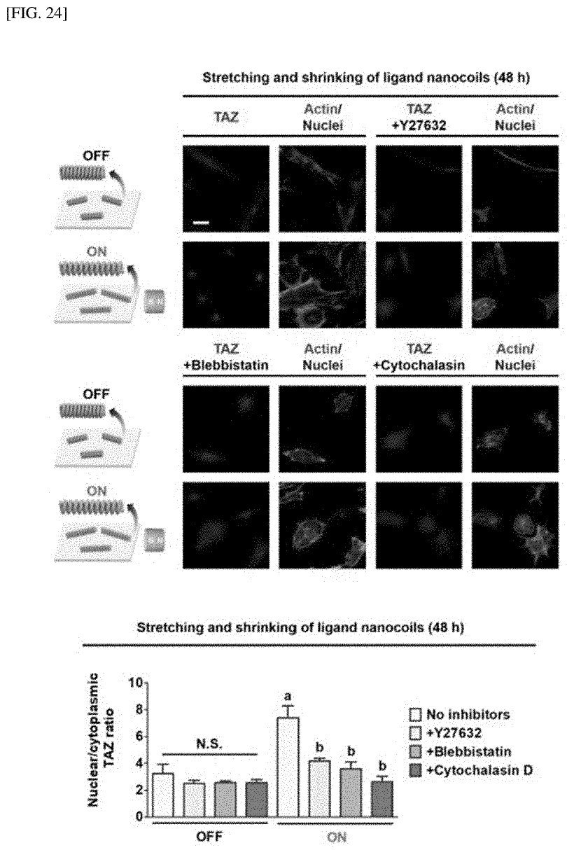

FIG. 24 is a confocal immunofluorescent image of TAZ, F-actin, and nuclei in stem cells cultured for 48 hours in a medium without an inhibitor and a medium with actin polymerization inhibitor (cytochalasin D), ROCK inhibitor (Y27632), and myosin II inhibitor (blebbistatin) by using the nanocoil-substrate complex according to the present invention, and a scale bar represents 50 μm (an upper part), and is a graph representing a result of a calculation of nuclear/cytoplasmic YAP fluorescence ratio by cytochalasin D, Y27632, and blebbistatin calculated from the confocal immunofluorescent image (a lower part).

Referring to FIGS. 22 to 24, it can be seen that mechanosensing of stem cells induced by the stretching (“ON”) mode in which the magnetic field is applied in the bimodal switching involves signaling molecules, such as myosin II, rho-associated protein kinase (ROCK), and actin polymerization, which positively regulate pronounced nuclear localization of YAP/TAZ mechanotransducers.

Through this, it can be seen that the case (“ON”) in which the magnetic field is applied in the bimodal switching mediates stem cell differentiation through YAP/TAZ mechanotransduction.

### Experimental Example 5

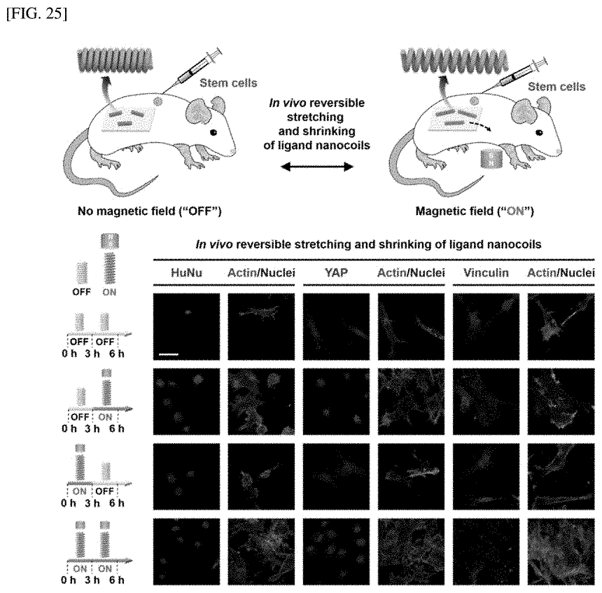

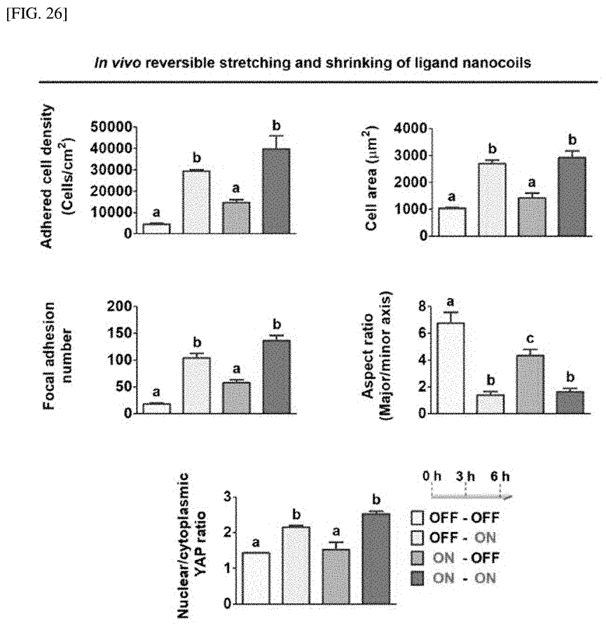

The experiment was performed to confirm the control of the adhesion and the mechanotransduction of stem cells in vivo for the stretching and the compression of the nanocoil according to the application of the magnetic field by using the nanocoil-substrate complex according to the present invention, and the result thereof is represented in FIGS. 25 and 26.

FIG. 25 is a result of an experiment for host stem cell adhesion control in vivo by using the nanocoil-substrate complex according to the present invention, and an upper part of FIG. 25 is an immunofluorescent confocal image of human-specific nuclear antigen (HuNu), F-actin, and nucleus in stem cells in the case of including the nanocoil with different magnetic field application order 6 hours after the injection of hMSC on the subcutaneous implanted substrate, and a scale bar is 50 μm.

FIG. 26 is a graph illustrating adherent cell density, cell area, focal adhesion number, aspect ratio (major axis/minor axis ratio), and nuclear/cytoplasm TAZ fluorescent ratio calculated from the confocal immunofluorescent image of FIG. 25.

A lower part of FIG. 25 is an image illustrating the experiment conducted by implementing the nanocoil-substrate complex according to the present invention into subcutaneous pockets of nude mice and then injecting hMSC.

Referring to FIGS. 25 and 26, it can be seen the injected hMSCs that had adhered to the substrate by co-localization of human-specific nuclear antigen (HuNu) and DAPI-positive nuclei in immunofluorescence of all cases in the bimodal switching. Further, immunofluorescence confirmed that in the reversible bimodal switching in vivo, the stretching (“OFF-ON” and “ON-ON” groups) group in which the magnetic field is applied stimulates significantly higher adherent density and focal adhesion of stem cells over a wider area and vinculin clustering, and YAP mechanotransduction, compared to the compression (“OFF”) group in which the magnetic field is removed, which also promoted the adhesion of host immune cells over prolonged time.

What is claimed is:

1. A nanocoil-substrate complex for controlling adhesion and differentiation of stem cells, the nanocoil-substrate complex comprising:
a substrate;
one or more nanocoils chemically coupled to the substrate; and
one or more integrin ligand peptides chemically coupled to the nanocoil,
wherein the nanocoil is formed of a spiral nanowire and includes one or more metal elements,
the nanocoil has a length of 100 nm to 20 μm, and
the nanocoil has a length reversibly changed depending on application/non-application of a magnetic field within a range of Equation 1 below,

|L₁−L₀|>10 nm [Equation 1]
in Equation 1, L₁ is a length of the nanocoil when the magnetic field is applied, and L₀ is a length of the nanocoil when the magnetic field is not applied.

2. The nanocoil-substrate complex of claim 1, wherein the metal element includes one or more elements among cobalt (Co), iron (Fe), and nickel (Ni).

3. The nanocoil-substrate complex of claim 1, wherein the nanowire is provided in a form of a wire having a circular cross-section, and has a diameter of 5 nm to 100 nm, and
an average length of a spiral outer diameter of the nanocoil is 50 nm to 200 nm.

4. The nanocoil-substrate complex of claim 1, wherein the applied magnetic field has a size of 100 mT to 7 T.

5. The nanocoil-substrate complex of claim 1, wherein a plurality of integrin ligand peptides is coupled to the nanocoil while being spaced apart from each other, and an average interval between the adjacent integrin ligands is 1 nm to 10 nm.

6. The nanocoil-substrate complex of claim 1, wherein when the magnetic field is applied, adjacent spirals of the nanocoil are spaced apart from each other, and a pitch between the adjacent spirals is 1 nm to 100 nm.

7. The nanocoil-substrate complex of claim 1, wherein the integrin ligand peptide includes a thiolated integrin ligand peptide, and
a thiol group of the integrin ligand peptide is coupled to the spiral nanocoil by a polyethylene glycol linker.

8. The nanocoil-substrate complex of claim 1, wherein the nanocoil is coupled to the substrate by coupling carboxylate to the nanocoil.

9. The nanocoil-substrate complex of claim 1, wherein the surface of the substrate, which is not coupled with the nanocoil, is inactivated.

10. A method of preparing a nanocoil-substrate complex for controlling adhesion and differentiation of stem cells, the method comprising:
preparing a nanocoil by electrodepositing a solution including one or more metal elements;
coupling a carboxylate substituent to the nanocoil by mixing the nanocoil and a first suspension;
manufacturing a substrate coupled with the nanocoil by soaking a substrate, of which a surface is activated, in a solution containing the nanocoil to which the carboxylate is coupled;
coupling a linker to a distal end of the nanocoil by soaking the substrate coupled with the nanocoil in a solution containing a polyethylene glycol linker; and
coupling an integrin ligand peptide (RGD) to the nanocoil by mixing a second suspension containing the integrin ligand peptide and the activated substrate coupled with the nanocoil.

11. The method of claim 10, wherein in the preparing of the nanocoil, the solution containing the metal element includes one or more elements among cobalt (Co), iron (Fe), and nickel (Ni).

12. The method of claim 10, wherein in the coupling of the carboxylate substituent, the first suspension includes an amino acid derivative containing a carboxylate substituent, and
the amino acid derivative is coupled to a surface of the nanocoil.

13. The method of claim 11, wherein in the coupling of the integrin ligand peptide, the second suspension includes thiolated integrin ligand peptide.

14. The method of claim 11, wherein the manufacturing of the substrate coupled with the nanocoil uses the substrate, of which the surface is aminated, by activating the surface of the substrate by immersing the substrate in an acid solution and then soaking the substrate, of which the surface is activated, in an aminosilane solution.

15. The method of claim 11, further comprising:
after the coupling of the integrin ligand peptide to the nanocoil, soaking the substrate coupled with the nanocoil in a solution including a polyethylene glycol derivative and inactivating a surface of the substrate which is not coupled with the nanocoil.

16. A method of controlling adhesion and differentiation of stem cells, the method comprising:
controlling cell adhesion and differentiation of stem cells by treating the nanocoil-substrate complex for controlling cell adhesion and differentiation of the stem cells according to claim 1 with a culture medium and then applying a magnetic field in a range from 20 mT to 7 T,
wherein the nanocoil has a length reversibly changed within Equation 1 below depending on application/non-application of the magnetic field,

|L₁−L₀|>10 nm [Equation 1]
in Equation 1, L₁ is a length of the nanocoil when the magnetic field is applied, and L₀ is a length of the nanocoil when the magnetic field is not applied.

17. The method of claim 16, wherein the controlling of the adhesion and the differentiation of the stem cells includes controlling the adhesion and the differentiation of the stem cells in vivo and ex vivo by reversibly changing the length of the nanocoil depending on the application/non-application of the magnetic field to the nanocoil-substrate complex.

18. The method of claim 16, wherein the adhesion and mechanosensing differentiation of stem cells are degraded in the case where the magnetic field is not applied to the nanocoil-substrate complex.

19. The method of claim 16, wherein the adhesion and mechanosensing differentiation of stem cells are promoted in the case where the magnetic field is applied to the nanocoil-substrate complex.
